(19) United States
(12) Patent Application Publication
(10) Pub. No.: US 2024/0318157 A1
(43) Pub. Date: Sep. 26, 2024

(54) Neurotensin Variants And Tagged Proteins Comprising Neurotensin Or Sortilin Propeptide

(71) Applicant: Amicus Therapeutics, Inc., Philadelphia, PA (US)

(72) Inventors: Helen Eisenach, Cranbury, NJ (US); Steven Tuske, Cranbury, NJ (US)

(73) Assignee: Amicus Therapeutics, Inc., Philadelphia, PA (US)

(21) Appl. No.: 18/575,512

(22) PCT Filed: Jul. 1, 2022
(86) PCT No.: PCT/US2022/073333

§ 371 Date: Dec. 29, 2023

Related U.S. Application Data
(60) Provisional application No. 63/217,500, filed on Jul. 1, 2021.

(51) Int. Cl. C12N 9/24; C07K 7/08; C07K 14/705

(52) U.S. Cl. CPC C12N 9/2402; C07K 7/083; C07K 14/70571; C12Y 302/0102; C07K 2319/06; C07K 2319/74

(57) ABSTRACT

The disclosure provides amino acid sequences for neurotensin variants as well as recombinant proteins that contain neurotensin, sortilin propeptide, or variants thereof. The disclosure also provides method of production and method for determining cellular uptake for the recombinant proteins. Gene therapy compositions, pharmaceutical compositions, methods of treatment, and uses of the gene therapy compositions and the recombinant proteins are also disclosed.

### TECHNICAL FIELD

The present invention relates to treating genetic disorders.

### BACKGROUND

Genetic disorders such as lysosomal storage disorders arise via heritable or de novo mutations occurring in gene coding regions of the genome. The mutations in the regions may result in defective proteins, which are crucial for cellular metabolic activities. Since many mutant proteins are unstable in the ER (Ishii et al., Biochem. Biophys. Res. Comm. 1996; 220: 812-815), the protein is retarded in the normal transport pathway (ER→Golgi apparatus→endosomes→lysosome) and prematurely degraded. The resulting protein deficiency may cause accumulation of toxic compounds and subsequent disruption of normal cellular functioning.

Protein replacement therapy is one of the approved treatments for treating the genetic disorders. The therapy typically involves intravenous infusion of a purified form of the corresponding wild-type protein. However, one of the main complications with protein replacement therapy is attainment and maintenance of therapeutically effective amounts of the protein in vivo due to low intracellular bioavailability, and rapid degradation and/or clearance of the infused protein. The current approach to overcome this problem is to perform numerous costly high dose infusions.

Gene therapy is another potential approach to treat the genetic disorders. Gene therapy involves replacing or supplementing the defective gene with a nucleic acid sequence that encodes a functional protein. Gene therapies may employ recombinant vectors to deliver nucleic acid sequences that encode a functional protein or genetically modified human cells that express a functional protein. However, delivery of the transgene or transgene product to the relevant target cells and organelles either by direct transduction or by cross-correction remains a challenge in gene therapy.

Accordingly, due to the challenges with reaching the relevant cell and organelles for both gene therapy and protein replacement therapy, there is a need for new therapies that provide more precise targeting for treating genetic disorders.

### SUMMARY

Neurotensin is a neuropeptide encoded by the neurotensin gene. In some embodiments, neurotensin functions as one or more of a hormone, a growth factor, an apoptotic signaling peptide and a lysosomal transport facilitator peptide. One of the natural receptors for neurotensin is sortilin (also referred to as Neurotensin Receptor 3, NTSR3), which is mainly present on Golgi apparatus membrane, nucleus membrane, lysosome membrane, cell plasma membrane, endoplasmic reticulum membrane and endosome membrane, and co-localizes with CI-MPR/IGF2R in the lysosome. Sortilin also has high levels of expression in specific tissues such as kidney, heart, brain, and reproductive tissue. Accordingly, various aspects of the present invention utilize neurotensin or variants thereof to provide precise targeting of therapeutic proteins to cells or organelles with high expression of sortilin.

Various aspects of the present invention relate to a variant of the wild-type neurotensin peptide amino acid sequence. In one or more embodiments, the neurotensin peptide amino acid sequence comprises at least 70% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13, such that the amino acid sequence is not SEQ ID NO:1. In one or more embodiments, the neurotensin peptide amino acid sequence comprises at least 90% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13, such that the amino acid sequence is not SEQ ID NO:1. In one or more embodiments, the neurotensin peptide amino acid sequence comprises SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

One or more embodiments of the present invention relate to a polynucleotide comprising a nucleotide sequence encoding a neurotensin peptide having at least 70% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13, such that the resulting amino acid sequence is not SEQ ID NO:1. In one or more embodiments, the nucleotide sequence encodes a neurotensin peptide having at least 90% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13, such that the resulting amino acid sequence is not SEQ ID NO:1. In one or more embodiments, the nucleotide sequence encodes a neurotensin peptide having SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

Various aspects of the present invention relate to a neurotensin polynucleotide comprising at least 40% nucleotide sequence identity to SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, such that the resulting polypeptide is not SEQ ID NO:1. In one or more embodiments, the polynucleotide has at least 70% nucleotide sequence identity to SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, such that the polynucleotide is not SEQ ID NO:14. In one or more embodiments, the polynucleotide has at least 97% nucleotide sequence identity to SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, such that the polynucleotide is not SEQ ID NO:14. One or more embodiments comprising a neurotensin polynucleotide having SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

Various aspects of the present invention relate to a tagged protein comprising a a therapeutic protein and one or more tags selected from a neurotensin peptide and/or a sortilin propeptide. In one or more embodiments, the neurotensin peptide of the tagged protein comprises a polypeptide that is at least 70% identical to SEQ ID NO: 1. In one or more embodiments, the neurotensin peptide of the tagged protein comprises a polypeptide that is at least 90% identical to SEQ ID NO: 1. In one or more embodiments, the neurotensin peptide of the tagged protein comprises SEQ ID NO: 1. In one or more embodiments, the neurotensin peptide of the tagged protein comprises a polypeptide that is at least 70% identical to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. In one or more embodiments, the neurotensin peptide of the tagged protein comprises a polypeptide that is at least 90% identical to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. In one or more embodiments, the neurotensin peptide of the tagged protein comprises SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13. In one or more embodiments, the sortilin propeptide of the tagged protein comprises a polypeptide that is at least 70% identical to SEQ ID NO: 48. In one or more embodiments, the sortilin propeptide of the tagged protein comprises a polypeptide that is at least 90% identical to SEQ ID NO: 48. In one or more embodiments, the sortilin propeptide of the tagged protein comprises SEQ ID NO: 48. In other embodiments, the sortilin propeptide is a variant that is at least 40%, 70% or 97% identical to SEQ ID NO: 48, but is not SEQ ID NO: 48.

In one or more embodiments, the therapeutic protein comprises a soluble protein. In one or more embodiments, the therapeutic protein comprises a lysosomal protein. In one or more embodiments, the therapeutic protein of the tagged protein comprises palmitoyl protein thioesterase 1 (PPT1) (CLN1), tripeptidyl peptidase 1 (TPP1) (CLN2), Cathepsin D (CTSD) (CLN10), progranulin (PGRN) (CLN11) and cathepsin F (CTSF) (CLN13), alpha-galactosidase A, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, or an enzymatically active fragment thereof. In one or more embodiments, the therapeutic protein of the tagged protein comprises a Batten-related protein selected from PPT1, TPP1, CTSD, PGRN or CTSF. In one or more embodiments, the therapeutic protein of the tagged protein comprises lysosomal alpha-glucosidase (GAA).

In one or more embodiments, the tagged protein comprises one or more of an affinity tag, a linker peptide, and a secretion signal peptide.

Various aspects of the present invention relate to a polynucleotide comprising a nucleotide sequence encoding the tagged protein. In one or more embodiments, the polynucleotide sequence comprises a therapeutic protein polynucleotide sequence and one or more tag sequences comprising a neurotensin polynucleotide sequence and/or a sortilin propeptide polynucleotide sequence. In one or more embodiments, the neurotensin polynucleotide sequence comprises at least 40% sequence identity to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26. In one or more embodiments, the neurotensin polynucleotide sequence comprises at least 70% sequence identity to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26. In one or more embodiments, the neurotensin polynucleotide sequence comprises at least 97% sequence identity to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26. In one or more embodiments, the neurotensin polynucleotide sequence comprises SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26. In one or more embodiments, the sortilin propeptide polynucleotide sequence comprises at least 40%, 70%, 97% or 100% to sequence identity to SEQ ID NO: 49.

Various aspects of the present invention relate to a method of producing the tagged protein. The method comprises expressing the tagged protein and purifying the tagged protein. In one or more embodiments, the tagged protein is expressed in Expi293F cells, PC12 cells, COS7 cell, HAP1 cells, Chinese hamster ovary (CHO) cells, HeLa cells, human embryonic kidney (HEK) cells, mouse primary myoblasts, NIH 3T3 cells, Escherichia coli cells, baculovirus expression system (e.g., Sf9 cells, Sf21 cells), yeast cells (e.g., Saccharomyces cerevisiae), or a variant thereof.

Various aspects of the present invention relate to a method of determining cellular uptake for the tagged protein. In one or more embodiments, the method comprises culturing cells, incubating the cells with the tagged protein, and identifying the intracellularly transported tagged protein. In one or more embodiments, the method may comprise determining functionality of the intracellularly transported tagged protein. In one or more embodiments, the method may comprise quantifying the intracellularly transported tagged protein. In one or more embodiments, the method is performed in Expi293F cells, PC12 cells, COS7 cell, HAP1 cells, Chinese hamster ovary (CHO) cells, HeLa cells, human embryonic kidney (HEK) cells, mouse primary myoblasts, NIH 3T3 cells, Escherichia coli cells, Sf9 cells, Sf21 cells, Saccharomyces cerevisiae or a variant thereof.

Various aspects of the present invention relate to a method of assessing sortilin binding efficiency for the tagged protein. In one or more embodiments, the method comprises incubating immobilized sortilin with the tagged protein and quantifying the proportion of sortilin-bound tagged protein. In one or more embodiments, the sortilin comprises a domain or a full-length protein. In one or more embodiments, the sortilin domain comprises amino acid residues 78-755 of the full length sortilin amino acid sequence. In one or more embodiments, the method comprises measuring functionality of the sortilin-bound tagged protein.

Various aspects of the present invention relate to a pharmaceutical formulation comprising the neurotensin peptide, tagged protein, and/or a polynucleotide encoding the neurotensin peptide or the tagged protein. The pharmaceutical formulation may also include a pharmaceutically acceptable carrier and/or one or more pharmaceutically acceptable excipients.

Various aspects of the present invention relate to a method of treating a disease or disorder comprising administering the pharmaceutical formulation to a patient in need thereof. In one or more embodiments, the disease or disorder is Fabry disease and the therapeutic protein comprises alpha-galactosidase A. In one or more embodiments, the disease or disorder is Pompe disease and the therapeutic protein comprises acid alpha-glucosidase. In one or more embodiments, the disease or disorder is Batten disease and the therapeutic protein comprises PPT1 (CLN1), TPP1 (CLN2), CTSD (CLN10), PGRN (CLN11), or CTSF (CLN13). In one or more embodiments, the pharmaceutical formulation is administered intrathecally, intravenously, intracisternally, intracerebroventrically, intraocularly, intravitreally, retinally, subretinally, intramuscularly, subcutaneously, intracerebrally, surgically or intraparenchymally.

Various aspects of the present invention relate to a gene therapy composition comprising a gene therapy delivery system and the polynucleotide encoding the tagged protein. In one or more embodiments, the gene therapy delivery system comprises one or more of a vector, a liposome, a lipid-nucleic acid nanoparticle, an exosome, and a gene editing system. In one or more embodiments, the gene therapy editing system comprises one or more of Clustered Regularly Interspaced Short Palindromic Repeat (CRISPR) associated protein 9 (CRISPR-Cas-9), Transcription activator-like effector nuclease (TALEN), or ZNF (Zinc finger protein). In one or more embodiments, the gene therapy delivery system comprises a viral vector. In one or more embodiments, the viral vector comprises one or more of an adenoviral vector, an adeno-associated viral vector, a lentiviral vector, a retroviral vector, a poxviral vector, or a herpes simplex viral vector. In one or more embodiments, the viral vector comprises a viral polynucleotide operably linked to the polynucleotide encoding the tagged protein. In one or more embodiments, the viral vector comprises at least one inverted terminal repeat (ITR). In one or more embodiments, the viral vector comprises one or more of an SV40 intron, a polyadenylation signal (e.g., Bovine growth hormone polyadenylation signal (bGHpolyA)), or a stabilizing element (e.g., Woodchuck Hepatitis Virus (WHP) Posttranscriptional Regulatory Element (WPRE)). In one or more embodiments, the gene therapy delivery system comprises a promoter (e.g., CBA). In one or more embodiments, the gene therapy delivery system comprises a polynucleotide encoding a secretion signal peptide (e.g., human BiP or a variant thereof). In one or more embodiments, the gene therapy composition comprises a pharmaceutically acceptable carrier.

Various aspects of the present invention relate to a method of treating a disease or disorder comprising administering the gene therapy composition to a patient in need thereof. In one or more embodiments, the disease or disorder is Fabry disease and the therapeutic protein comprises alpha-galactosidase A. In one or more embodiments, the disease or disorder is Pompe disease and the therapeutic protein comprises alpha-glucosidase. In one or more embodiments, the disease or disorder is Batten disease and the therapeutic protein comprises PPT1 (CLN1), TPP1 (CLN2), CTSD (CLN10), PGRN (CLN11), or CTSF (CLN13). In one of more embodiments, the gene therapy composition is administered intrathecally, intravenously, intracisternally, intracerebroventrically, intraocularly, intravitreally, retinally, subretinally, intramuscularly, subcutaneously, intracerebrally, surgically or intraparenchymally.

### BRIEF DESCRIPTION OF DRAWINGS

FIGS. 1A-1B. The binding efficiency of the GAA tagged protein for sortilin. Two variants of purified GAA tagged proteins, the GAA tagged protein with C-terminal NT tag (GAA-NT) (FIG. 1A) and the GAA tagged protein with N-terminal NT tag (NT-GAA) (FIG. 1B, were co-immunoprecipitated separately with soluble sortilin domain using a commercially available co-immunoprecipitation kit (Thermo Fisher, 26149). The images show that both GAA-NT and NT-GAA have high affinity for the soluble sortilin domain.

FIG. 2. α-D-glucopyranoside activity of sortilin-bound GAA tagged protein. Extracellular soluble sortilin domain with a C-terminal TwinStrep tag for immobilized on a 96-well microplate. The wells were then incubated with varying concentrations of NT-GAA (GAA tagged protein with N-terminal NT tag), GAA-NT (GAA tagged protein with C-terminal NT tag) or GAA (GAA with no NT tag). A fluorogenic 4-methylumbelliferyl-α-D-glucopyranoside substrate was used to measure α-D-glucopyranoside activity of the sortilin-bound tagged proteins. FIG. 2 shows α-D-glucopyranoside activity of sortilin-bound NT-GAA and GAA-NT. Wells incubated with GAA without the NT tag had no α-D-glucopyranoside activity indicating that the protein did not bind to sortilin.

FIGS. 3A-3B. α-D-glucopyranoside activity of intracellularly transported GAA tagged protein. Uptake of GAA tagged proteins was tested in HAP1 KO cells by determining the α-D-glucopyranoside activity of intracellularly transported GAA tagged protein. The uptake assay for NT-GAA (GAA tagged protein with N-terminal NT tag), GAA-NT (GAA tagged protein with C-terminal NT tag) or GAA (GAA with no NT tag) was performed in the presence and absence of 10 mM mannose-6-phosphate (M6P). Following the uptake assay, the cells were lysed and α-D-glucopyranoside activity was measured using a fluorogenic substrate 4-methylumbelliferyl-α-D-glucopyranoside at pH 4.8. FIG. 3A shows α-D-glucopyranoside activity for NT-GAA, GAA-NT and GAA. Similarly, the western blot analysis in FIG. 3B shows intracellularly transported NT-GAA, GAA-NT and GAA in the presence of M6P.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Various embodiments of the present invention relate to a method for delivering a therapeutic molecule to a cell. In one or more embodiments, the method is used to deliver a therapeutic protein for a genetic disorder.

### Definitions

The term “Batten-related proteins” refers to proteins implicated in Batten disease such as ceroid lipofuscinosis neuronal proteins. Batten disease includes CLN1, CLN2, CLN3, CLN4, CLN5, CLN6, CLN7, CLN8, CLN10, CLN11, CLN12, CLN13 and CLN14. Non-limiting, exemplary ceroid lipofuscinosis neuronal proteins and corresponding Batten diseases are shown in Table 1.

TABLE 1
Batten Disease Proteins

| Disease | Protein | Solubility |
|---|---|---|
| CLN1 | PPT1, palmitoyl protein thioesterase 1; | Soluble |
| CLN2 | TPP1, tripeptidyl peptidase 1 | Soluble |
| CLN3 | CLN3, ceroid lipofuscinosis neuronal 3 | Membrane |
| CLN4 | DNAJC5, DnaJ heat shock | Membrane |
| | protein family member C5 | |
| CLN5 | CLN5, ceroid lipofuscinosis neuronal 5 | Membrane |
| CLN6 | CLN6, ceroid lipofuscinosis neuronal 6 | Membrane (ER) |
| CLN7 | MFSD8, major facilitator | Membrane |
| | superfamily domain-containing 8 | |
| CLN8 | CLN8, ceroid lipofuscinosis neuronal 8 | Membrane (ER) |
| CLN10 | CTSD, cathepsin D | Soluble |
| CLN11 | PGRN, progranulin | Soluble |
| CLN12 | ATP13A2 | Membrane |
| CLN13 | CTSF, cathepsin F | Soluble |
| CLN14 | KCTD7, potassium channel | Membrane |
| | tetramerization domain-containing 7 | |

In one or more embodiments, the ceroid lipofuscinosis neuronal proteins includes a soluble protein. Example of such soluble includes ceroid-lipofuscinosis neuronal protein 1 (Palmitoyl protein thioesterase such as Palmitoyl protein thioesterase 1 (PPT1)), ceroid-lipofuscinosis neuronal protein 2 (Tripeptidyl peptidase 1 (TPP1)), ceroid-lipofuscinosis neuronal protein 10 (Cathepsin D (CTSD)), ceroid-lipofuscinosis neuronal protein 11 (Progranulin (PGRN)), and ceroid-lipofuscinosis neuronal protein 13 (Cathepsin F (CTSF).

The term “tagged protein” refers to a protein comprising a tag and a therapeutic protein. In one or more embodiments, the tag comprises a neurotensin peptide and/or a sortilin propeptide. In one or more embodiments, the tagged protein may comprise at least one additional protein, peptide, or polypeptide, linked to it. The additional protein, peptide, or polypeptide may include one or more of a secretion signal peptide, a linker peptide, and an affinity tag.

The term “gene therapy delivery system” refers to any system that can be used to deliver an exogenous gene of interest to a target cell so that the gene of interest will be expressed or overexpressed in the target cell. In one or more embodiments, the target cell is an in vivo patient cell. In one or more embodiments, the target cell is an ex vivo cell and the cell is then administered to the patient.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

The term “minigene” refers to the combination of the transgene, promoter/enhancer, and 5′ and 3′ AAV ITRs is referred to as a “minigene” for ease of reference herein. Provided with the teachings of this invention, the design of such a minigene can be made by resort to conventional techniques.

The wildtype neurotensin peptide (NT) consists of 13 amino acids and is in GeneBank Accession no. P30990. In one or more embodiments, the wildtype neurotensin peptide may have one of more deletions, additions and/or substitutions. Non-limiting, exemplary neurotensin peptides and polynucleotide sequences encoding the same are provided in Table 2 below:

TABLE 2
Neurotensin Peptides and Polynucleotide Sequences

| Neurotensin | Amino Acid | SEQ ID | Polynucleotide | SEQ ID |
|---|---|---|---|---|
| Peptide | Sequence | NO: | Sequence | NO: |
| Wild-Type | ELYENKPRRPYIL | 1 | GAGCTGTACGAGAACAAGCC | 14 |
| NT | | | CAGACGCCCCTACATCCTG | |
| NT Variant | ELYENKPRRPFIL | 2 | GAGCTGTACGAGAACAAGCC | 15 |
| 1 | | | CAGACGCCCCTTCATCCTG | |
| NT Variant | ELYENKPRRPYIN | 3 | GAGCTGTACGAGAACAAGCC | 16 |
| 2 | | | CAGACGCCCCTACATCAAC | |
| NT Variant | ELYENKPRRPYID | 4 | GAGCTGTACGAGAACAAGCC | 17 |
| 3 | | | CAGACGCCCCTACATCGAC | |
| NT Variant | ELYENKPRRPYIE | 5 | GAGCTGTACGAGAACAAGCC | 18 |
| 4 | | | CAGACGCCCCTACATCGAG | |
| NT Variant | TLYENKPRRPYIL | 6 | ACCCTGTACGAGAACAAGCC | 19 |
| 5 | | | CAGACGCCCCTACATCCTG | |
| NT Variant | PLYENKPRRPYIL | 7 | CCCCTGTACGAGAACAAGCC | 20 |
| 6 | | | CAGACGCCCCTACATCCTG | |
| NT Variant | ELYEDKPRRPYIL | 8 | GAGCTGTACGACAACAAGCC | 21 |
| 7 | | | CAGACGCCCCTACATCCTG | |
| NT Variant | ELRENKPRRPYIL | 9 | GAGCTGAGAGAGAACAAGCC | 22 |
| 8 | | | CAGACGCCCCTACATCCTG | |
| NT Variant | ELYENKPRRGYIL | 10 | GAGCTGTACGAGAACAAGCC | 23 |
| 9 | | | CAGACGCGGCTACATCCTG | |
| NT Variant | ELYENKPRRPIIL | 11 | GAGCTGTACGAGAACAAGCC | 24 |
| 10 | | | CAGACGCCCCATCATCCTG | |
| NT Variant | ELYENKPARPYIL | 12 | GAGCTGTACGAGAACAAGCC | 25 |
| 11 | | | CGCCCGCCCCTACATCCTG | |
| NT Variant | ELYENKPRAPYIL | 13 | GAGCTGTACGAGAACAAGCC | 26 |
| 12 | | | CAGAGCCCCCTACATCCTG | |

Non-limiting, exemplary mutant sequences include SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. In one or more embodiments, NT may have at least 50%, at least 55%, at least 60%, at least 65%, at least 70%, at least 75%, at least 80%, at least 85%, or at least 90% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. One skilled in the art can readily derive a polynucleotide sequence encoding an amino acid sequence. Also, the polynucleotide sequence can be codon optimized for expression in target cells using commercially available products. In one or more embodiments, a polynucleotide sequence encoding NT may have at least 40%, at least 45%, at least 50%, at least 55%, at least 60%, at least 65%, at least 70%, at least 75%, at least 80%, at least 85%, at least 90%, at least 95%, or at least 97% sequence identity to SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, such that the resulting polypeptide is not SEQ ID NO:1. In one or more embodiments, the nucleotide sequence for the neurotensin peptide may comprise SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

The term “operatively linked” refers to the functional relationship of a polynucleotide/gene with regulatory and effector sequences of nucleotides, such as promoters, enhancers, transcriptional and translational stop sites, and other signal sequences. For example, operative linkage of a nucleic acid to a promoter refers to the physical and functional relationship between the polynucleotide and the promoter such that transcription of DNA is initiated from the promoter by an RNA polymerase that specifically recognizes and binds to the promoter. The promoter directs the transcription of RNA from the polynucleotide.

The term “patient” refers to any mammalian subject for whom diagnosis, treatment, or therapy is desired, particularly humans. The mammalian subject for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. The mammalian subject may be a fetus, a neonate, child, juvenile or an adult with disorder. In one or more embodiments, the mammalian subject is human.

The term “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

The term “promoter” refers to a DNA sequence to which the enzyme RNA polymerase binds and initiates the transcription of a DNA sequence into RNA. A promoter can be a synthetically or naturally synthesized, which is capable of conferring, activating or enhancing expression of a nucleic acid sequence in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include but not limited to CBA, P546, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, actin promoter, the myosin promoter, the elongation factor-1a promoter, the hemoglobin promoter, and the creatine kinase promoter, SV40 late promoter, SV40 early promoter, RSV-LTR promoter, CMV IE promoter, SV40 early promoter or SV40 late promoter and the CMV IE promoter.

The term “protein replacement therapy” is intended to refer to the introduction of an exogenous, purified protein into an individual having a deficiency in such protein. The administered protein can be obtained from natural sources or by recombinant expression. The term also refers to the introduction of a purified protein in an individual otherwise requiring or benefiting from administration of a purified protein. In at least one embodiment, such an individual suffers from protein deficiency. The term “protein deficiency” as used herein refers to one or more of a functional deficiency and a dietary deficiency. In one or more embodiments, the protein deficiency is a functional deficiency. The introduced protein may be a purified, recombinant protein produced in vitro, in a host cell, or a protein purified from isolated tissue or fluid, such as, for example, placenta or animal milk, or from plants.

The term “serotype” is a distinction with respect to an AAV having a capsid which is serologically distinct from other AAV serotypes. Serologic distinctiveness is determined on the basis of the lack of cross-reactivity between antibodies to the AAV as compared to other AAV.

Cross-reactivity is typically measured in a neutralizing antibody assay. For this assay polyclonal serum is generated against a specific AAV in a rabbit or other suitable animal model using the adeno-associated viruses. In this assay, the serum generated against a specific AAV is then tested in its ability to neutralize either the same (homologous) or a heterologous AAV. The dilution that achieves 50% neutralization is considered the neutralizing antibody titer. If for two AAVs the quotient of the heterologous titer divided by the homologous titer is lower than 16 in a reciprocal manner, those two vectors are considered as the same serotype. Conversely, if the ratio of the heterologous titer over the homologous titer is 16 or more in a reciprocal manner the two AAVs are considered distinct serotypes.

The term “sortilin” refers a membrane protein which is a single-pass transmembrane protein with a Vps10p extracellular domain. Sortilin is also referred to as Neurotensin Receptor 3 (NTSR3). The residues 78-755 forms the extracellular domain. Sortilin has high levels of expression in specific tissues, among which kidney, heart, brain, and reproductive tissues are the highest. At the cellular level, sortilin is mainly present on the Golgi apparatus membrane, nucleus membrane, lysosome membrane, cell plasma membrane, endoplasmic reticulum membrane and endosome membrane. Sortilin is known to co-localize with CI-MPR/IGF2R in the lysosome and mediates mannose-6-phosphate independent uptake of lysosomal proteins, e.g., alpha galactosidase (GLA), acid sphingomyelinase (ASM) etc. In some embodiments, neurotensin is engineered to interact with sortilin. In some embodiments, based on known crystal structures of sortilin in complex with NT (PBD: 3f6k, 4po7) in silico modeling, 12 NT variants, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13, were predicted to improve sortilin binding affinity for NT.

Non-limiting, exemplary therapeutic protein includes lysosomal proteins, such as alpha-glucosidase (GAA). Non-limiting, exemplary therapeutic proteins comprise PPT1 (CLN1), TPP1 (CLN2), CTSD (CLN10), PGRN (CLN11), CTSF (CLN13), alpha-galactosidase, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, or an enzymatically active fragment thereof.

The term “treating” refers to administering an agent, or carrying out a procedure, for the purposes of obtaining a therapeutic effect, including inhibiting, attenuating, reducing, preventing or altering at least one aspect or marker of a disorder, in a statistically significant manner or in a clinically significant manner. The term “treat” does not state or imply a cure for the underlying condition, rather includes: (a) preventing the disorder or a symptom of a disorder from occurring in a patient which may be predisposed to the disorder but has not yet been diagnosed as having it (e.g., including disorders that may be associated with or caused by a primary disorder); (b) inhibiting the disorder (i.e., arresting its development); (c) relieving the disorder (i.e., causing regression of the disorder); and (d) improving at least one symptom of the disorder. Treating may refer to any indicia of success in the treatment or amelioration or prevention of a disorder, including any objective or subjective parameter such as abatement; remission; diminishing of symptoms or making the disorder condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the compounds or agents of the present invention to prevent or delay, to alleviate, or to arrest or inhibit development of the symptoms or conditions associated with the disorder.

The term “vector” refers to gene therapy delivery vehicles, or carriers, that deliver therapeutic genes to cells. A vector is any vector suitable for use in gene therapy, e.g., any vector suitable for the therapeutic delivery of nucleic acid polymers (encoding a polypeptide or a variant thereof) into target cells of a patient. In some embodiments, the gene therapy vector delivers the nucleic acid encoding a tagged protein to a cell where the tagged protein is expressed and secreted from the cell. The vector may be of any type, for example it may be a plasmid vector or a minicircle DNA. Typically, the vector is a viral vector. The viral vector may, for example, be derived from an adeno-associated virus (AAV), a retrovirus, a lentivirus, a herpes simplex virus, or an adenovirus. The viral vectors also may include additional natural or synthetic elements for increasing expression and/or stabilizing the vector such as promoters (e.g., hybrid CBA promoter (CBh) and human synapsin 1 promoter (hSyn1)), a polyadenylation signals (e.g., Bovine growth hormone polyadenylation signal (bGHpolyA)), stabilizing elements (e.g., Woodchuck Hepatitis Virus (WHP) Posttranscriptional Regulatory Element (WPRE)) and/or an SV40 intron. In one or more embodiments, a vector may comprise a polynucleotide sequence flanking by regions that promote homologous recombination at a desired site in the genome, thus providing for expression of the desired protein (See Koller and Smithies, 1989, Proc. Natl. Acad. Sci. USA, 86:8932-8935; Zijlstra et al., 1989, Nature 342:435-438; U.S. Pat. No. 6,244,113 to Zarling et al.; and U.S. Pat. No. 6,200,812 to Pati et al.).

### AAV Vectors

### AAV Vectors to Deliver Transgenes

In various embodiments, the gene therapy compositions and/or methods utilize AAV vectors. Alternatively, other viral vectors or gene therapy delivery systems as described herein may be used.

AAV vectors from one of a number of clades can be utilized to deliver the transgene in vivo for transduction, and a number of examples are provided in detail in U.S. Pat. No. 7,198,951, which is incorporated herein in its entirety. Using the genome of these AAV vectors and the manufacturing process described herein, and those available in the art, a recombinant AAV (rAAV) can be generated as a vector for delivery of one or more of the transgenes provided herein.

### I. Clades

A clade is a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence.

The Neighbor-Joining algorithm has been described extensively in the literature. See, e.g., M. Nei and S. Kumar, Molecular Evolution and Phylogenetics (Oxford University Press, New York (2000)). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades.

While the clades defined herein are based primarily upon naturally occurring AAV vp1 capsids, the clades are not limited to naturally occurring AAV. The clades can encompass non-naturally occurring AAV, including, without limitation, recombinant, modified or altered, chimeric, hybrid, synthetic, artificial, etc., AAV which are phylogenetically related as determined using a Neighbor-Joining algorithm at least 75% (of at least 1000 replicates) and a Poisson correction distance measurement of no more than 0.05, based on alignment of the AAV vp1 amino acid sequence.

The clades described herein include Clade A (represented by AAV1 and AAV6), Clade B (represented by AAV2) and Clade C (represented by the AAV2-AAV3 hybrid), Clade D (represented by AAV7), Clade E (represented by AAV8), and Clade F (represented by human AAV9).

Clade B (AAV2) and Clade C (the AAV2-AAV3 hybrid) are the most abundant of those found in humans (22 isolates from 12 individuals for AAV2 and 17 isolates from 8 individuals for Clade C).

### Clade A (Represented by AAV1 and AAV6)

AAV vectors can include those of Clade A, which includes AAV1 and AAV6. See, e.g., International Publication No. WO 00/28061, 18 May 2000; Rutledge et al, J Virol, 72(1):309-319 (January 1998). In addition, this clade contains AAV described in U.S. Pat. No. 7,198,951.

### Clade B (Represented by AAV2 Clade)

In other embodiments, the AAV vectors include those of Clade B, including AAV2 and those described in U.S. Pat. No. 7,198,951. In one or more embodiments, one or more of the members of this clade has a capsid with an amino acid identity of at least 85% identity, at least 90% identity, at least 95% identity, or at least 97% identity over the full-length of the vp1, the vp2, or the vp3 of the AAV2 capsid.

### Clade C (Represented by AAV2-AAV3 Hybrid Clade)

In another aspect, the AAV vectors includes those of Clade C, which is characterized by containing AAV that are hybrids of the previously published AAV2 and AAV3, and those described in U.S. Pat. No. 7,198,951. In one embodiment, one or more of the members of this clade has a capsid with an amino acid identity of at least 85% identity, at least 90% identity, at least 95% identity, or at least 97% identity over the full-length of the vp1, the vp2, or the vp3 of the hu.4 and/or hu.2 capsid.

### Clade D (AAV7 Clade)

In another embodiment, the AAV vectors include those of Clade D, which includes AAV7 those described in U.S. Pat. No. 7,198,951. In one or more embodiments, one or more of the members of this clade has a capsid with an amino acid identity of at least 85% identity, at least 90% identity, at least 95% identity, or at least 97% identity over the full-length of the vp1, the vp2, or the vp3 of the AAV7 capsid.

### Clade E (Represented by AAV8 Clade)

In one aspect, the AAV vectors are those of Clade E, including AAV8 and those described in U.S. Pat. No. 7,198,951. In one or more embodiments, one or more of the members of this clade has a capsid with an amino acid identity of at least 85% identity, at least 90% identity, at least 95% identity, or at least 97% identity over the full-length of the vp1, the vp2, or the vp3 of the AAV8 capsid. In another embodiment, the invention provides novel AAV of Clade E as described in US Published Patent Application No. US 2003/0138772 A1 (Jul. 24 2003).

### Clade F (Represented by AAV 9 Clade)

In another embodiment of the invention, the AAV vectors include those Clade F, including AAV9 and those described in U.S. Pat. No. 7,198,951. In one or more embodiments, one or more of the members of this clade has a capsid with an amino acid identity of at least 85% identity, at least 90% identity, at least 95% identity, or at least 97% identity over the full-length of the vp1, the vp2, or the vp3 of the AAV9 capsid.

The AAV clades are useful for a variety of purposes, including providing ready collections of related AAV for generating viral vectors, and for generating targeting molecules. These clades may also be used as tools for a variety of purposes that will be readily apparent to one of skill in the art.

### Transgene

The transgene is a nucleic acid sequence, heterologous to the vector sequences flanking the transgene, which encodes a polypeptide, protein, or other product, of interest. The nucleic acid coding sequence can be operatively linked to regulatory components in a manner which permits or modulates transgene transcription, translation, and/or expression in a host cell.

The transgene may be used to correct or ameliorate gene deficiencies, which may include deficiencies in which normal genes are expressed at less than normal levels or deficiencies in which the fully functional gene product is not expressed. The transgene may be used to suppress gene expression in a specific cell, where gene expression is toxic or deleterious (e.g., using miR for suppressing gene expression in dorsal root ganglia). Alternatively, the transgene may provide a product to a cell which is not natively expressed in the cell type or in the host. A preferred type of transgene sequence encodes a therapeutic protein or polypeptide which is expressed in a host cell. The invention further includes using multiple transgenes. In certain situations, a different transgene may be used to encode each subunit of a protein, or to encode different peptides or proteins. This is desirable when the size of the DNA encoding the protein subunit is large. In order for the cell to produce a multi-subunit protein, the cell is transfected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., M. L. Donnelly, et al, J. Gen. Virol., 78(Pt 1):13-21 (January 1997); Furler, S., et al, Gene Ther., 8(11):864-873 (June 2001); Klump H., et al., Gene Ther., 8(10):811-817 (May 2001). This 2A peptide is significantly smaller than an IRES, making it well suited for use when space is a limiting factor. More often, when the transgene is large, consists of multi-subunits, or two transgenes are co-delivered. In some embodiments, rAAV carrying the desired transgene(s) or subunits can be co-administered to allow them to concatemerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

### Therapeutic Transgenes

In some embodiments, gene products, such as enzymes, may be useful in enzyme replacement therapy. Enzyme replacement therapy is useful in a variety of conditions resulting from deficient activity of enzyme. In some embodiments, a suitable gene includes a polynucleotide sequence encoding β-glucuronidase.

In some embodiments, gene products include non-naturally occurring polypeptides, such as chimeric or hybrid polypeptides having a non-naturally occurring amino acid sequence containing insertions, deletions or amino acid substitutions.

### NT and Variants

The amino acid sequence of the wild-type NT is provided in SEQ ID NO: 1. Various embodiments of the present invention provide novel NT variants. The amino acid sequences of the NT variants are provided in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. In one or more embodiments, the NT amino acid sequence comprises at least 50%, at least 55%, at least 60%, at least 65%, at least 70%, at least 75%, at least 80%, at least 85%, at least 90%, or at least 95% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. In one or more embodiments, the NT amino acid sequence may comprise one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13.

In one or more embodiments, a polynucleotide comprises a nucleotide sequence such that resulting polypeptide has at least 50%, at least 55%, at least 60%, at least 65%, at least 70%, at least 75%, at least 80%, at least 85%, at least 90%, or at least 95% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. In one or more embodiments, a polynucleotide comprises a nucleotide sequence encoding SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13.

Similarly, in one of more embodiments, a neurotensin polynucleotide encoding NT comprises at least 40%, at least 45%, at least 50%, at least 55%, at least 60%, at least 65%, at least 70%, at least 75%, at least 80%, at least 85%, at least 90%, at least 95%, or at least 97% nucleotide sequence identity to SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, such that the resulting polypeptide is not SEQ ID NO:1. In one of more embodiments, a polynucleotide encoding NT comprises SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

Sortilin has two possible interaction sites for NT, one interaction site with the amino-terminus of NT, and one interaction site with the carboxy-terminus. Accordingly, a tagged protein comprising NT and a therapeutic protein can be cloned into an appropriate plasmid. In some embodiments, when expressed, the therapeutic protein is either on the amino or carboxy terminus of NT. In one or more embodiments, the NT may comprise the amino acid sequence that is at least 50%, at least 55%, at least 60%, at least 65%, at least 70%, at least 75%, at least 80%, at least 85%, at least 90%, at least 95%, or 100% similar to SEQ ID NO: 1. In one of more embodiments, the NT may comprise the amino acid having at least 5, at least 4, at least 3, at least 2 or at least one mutation, addition or deletion to SEQ ID NO:1. In one or more embodiments, the NT amino acid sequence comprise of at least 50%, at least 55%, at least 60%, at least 65%, at least 70%, at least 75%, at least 80%, at least 85%, at least 90%, at least 95%, or 100% sequence similarity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. In one or more embodiments, a polynucleotide comprising nucleotide sequence such that the resulting polypeptide has at least 50%, at least 55%, at least 60%, at least 65%, at least 70%, at least 75%, at least 80%, at least 85%, at least 90%, at least 95%, or 100% sequence similarity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. In one or more embodiments, a polynucleotide sequence encoding NT comprises at least 40%, at least 45%, at least 50%, at least 55%, at least 60%, at least 65%, at least 70%, at least 75%, at least 80%, at least 85%, at least 90%, at least 95%, at least 97%, or 100% sequence similarity to SEQ ID NO: 14, SEQ ID NO: 15, SEQ ID NO: 16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26.

### Sortilin Propeptide

Sortilin is synthesized with a sortilin propeptide. The sortilin propeptide gets cleaved off during maturation of sortilin. The sortilin propeptide has a high binding affinity for a matured sortilin. Accordingly, in one or more embodiments, a tagged protein comprising the sortilin propeptide and a therapeutic protein can be cloned into an appropriate plasmid. In some embodiments, when expressed, the therapeutic protein is on the amino and/or carboxy terminus of the sortilin propeptide.

In one or more embodiments, the sortilin propeptide comprises the amino acid sequence that is at least 50%, at least 55%, at least 60%, at least 65%, at least 70%, at least 75%, at least 80%, at least 85%, at least 90%, at least 95%, or 100% similar to SEQ ID NO: 48. In one of more embodiments, the NT may comprise the amino acid sequence having at least 5, at least 4, at least 3, at least 2 or at least one mutation, addition or deletion to SEQ ID NO: 48. In one or more embodiments, a polynucleotide comprising nucleotide sequence such that the resulting polypeptide has at least 50%, at least 55%, at least 60%, at least 65%, at least 70%, at least 75%, at least 80%, at least 85%, at least 90%, at least 95%, or 100% sequence similarity to SEQ ID NO: 48. In one or more embodiments, a polynucleotide sequence encoding the sortilin propeptide comprises at least 40%, at least 45%, at least 50%, at least 55%, at least 60%, at least 65%, at least 70%, at least 75%, at least 80%, at least 85%, at least 90%, at least 95%, at least 97%, or 100% sequence similarity to SEQ ID NO: 49.

In some embodiments, the sortilin propeptide's binding affinity for sortilin is more or equal to the NT's binding affinity for sortilin.

### Tagged Proteins

One aspect of the present invention relates to tagged proteins comprising a therapeutic protein and a tag. In some embodiments, the tag can be present on the amino and/or carboxy terminus. In one or more embodiments, the tag comprises the neurotensin peptide and/or the sortilin propeptide. In some embodiments, the tag comprises sortilin propeptide on amino terminus and the neurotensin on carboxy terminus. In some embodiments, the tag comprises the neurotensin on amino terminus and the sortilin propeptide on carboxy terminus. In one or more embodiments, the tagged protein comprises the neurotensin and/or sortilin propeptide. In some embodiments, the tagged protein comprises the neurotensin on amino terminus and the sortilin propeptide on carboxy terminus. In some embodiments, the tagged protein comprises the sortilin propeptide on amino terminus and the neurotensin on carboxy terminus.

In one or more embodiments, the therapeutic protein comprises a lysosomal protein. In one or more embodiments, the therapeutic protein comprises alpha-galactosidase, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, and other Batten-related proteins, or an enzymatically active fragment thereof. In one or more embodiments, the Batten-related protein comprises PPT1 (CLN1), TPP1 (CLN2), CTSD (CLN10), PGRN (CLN11), or CTSF (CLN13).

In one or more embodiments, the therapeutic protein comprises non-naturally occurring polypeptides, such as chimeric or hybrid polypeptides having a non-naturally occurring amino acid sequence containing insertions, deletions or amino acid substitutions.

In some embodiments, the tagged protein comprises one or more additional amino acid sequences encoding one or more functional domains or sequences, including one or more of the other functional domains and sequences provided herein. Exemplary functional domains or sequences include, but are not limited to, an affinity tag, a linker peptide, protease cleavage sites, a secretion signal peptide, cellular targeting domains, reporters, enzymes, or combination thereof. In one or more embodiments, the tagged protein comprises an affinity tag, wherein the affinity tag is linked to the therapeutic protein such that NT's terminal end(s) remains free. In some embodiments, the affinity tag may be one or more of mCherry, TwinStrep, polyhistidine, HA, FLAG, GST, and GFP.

In some embodiments, proteolytic cleavage sites may be engineered into the tagged protein to promote the release of the protein of interest from the vesicular targeting protein and/or other peptide functional domains, including affinity tags, in conjunction with tagged protein synthesis or purification. Exemplary protease cleavage sites include, but are not limited to, cleavage sites sensitive to thrombin, furin, factor Xa, metalloproteases, enterokinases, and cathepsin.

The cellular targeting domain may comprise amino acid sequences conferring cell-type specific or cell differentiation-specific targeting.

Functional domains in the tagged proteins of the present invention may be separated from one another by a spacer or linker to facilitate the independent folding of each peptide portion relative to one another and ensure that the individual peptide portions in a tagged protein do not interfere with one another. The spacer may include any amino acid or mixtures thereof. Preferably, a chosen spacer will increase the flexibility of the protein and facilitate adoption of an extended conformation. Preferred peptide spacers are comprised of the amino acids proline, lysine, glycine, alanine, and serine, and combinations thereof. In one embodiment, the linker is a glycine rich linker.

Linker or spacer sequences comprise 4 to 15 amino acids in length. Suitable amino acids for incorporation in linkers are alanine, arginine, serine or glycine. In some embodiments, the linker sequence comprises a lysosomal cleavage sequence. In some embodiments, the lysosomal cleavage sequence comprises an amino acid according to SEQ ID NO: 27. In one or more embodiments, the tagged protein comprises a linker peptide between the affinity tag and the therapeutic protein. In one or more embodiments, the tagged protein comprises a linker peptide between the tag (e.g., neurotensin peptide or sortilin propeptide) and the therapeutic protein. In some embodiments, the linker peptide comprise repeated glycine residues, repeated glycine-serine residues, or combinations thereof. In some embodiments, the linker peptide comprises 5-20 amino acids, 5-15 amino acids, 5-10 amino acids or 8-12 amino acids. In some embodiments, the linker peptide comprises about 5, 6, 7, 8, 9, 10, 11, 12 or 13 amino acids. Suitable linkers for gene therapy and enzyme replacement therapy constructs herein include but are not limited to those provided in Table 3 below.

TABLE 3
Linker Sequences

| GS Linkers | Sequence | SEQ ID NO: |
|---|---|---|
| | GGGGSGGGG | 28 |
| | GGGGS | 29 |
| | GGGSGGGGS | 30 |
| | GGGGSGGGS | 31 |
| | GGSGSGSTS | 32 |
| | GGGGSGGGGS | 33 |
| | GGGGSGSGGGGS | 34 |
| Lysosomal cleavage | RPRAVPTQA | 35 |
| linker | | |

In some embodiments, linkers for gene therapy and/or enzyme replacement therapy constructs comprises amino acid sequence according to SEQ ID NO: 28, SEQ ID NO: 29, SEQ ID NO: 30, SEQ ID NO: 31, SEQ ID NO: 32, SEQ ID NO: 33, SEQ ID NO: 34 or SEQ ID NO: 35.

In one or more embodiments, the tagged protein may comprise a secretion signal peptide. The signal peptide may be derived from a lysosomal protein including but not limited to PPT1, TPP1, GAA, GLA, NAGLU, SGSH or any other lysosomal enzyme that contains a signal peptide. Exemplary signal peptides are listed in Table 4 below:

TABLE 4
Signal Peptide Sequences

| Signal Peptide | Amino Acid Sequence | SEQ ID NO: |
|---|---|---|
| Native human BiP | MKLSLVAAMLLLLSAARA | 36 |
| Modified BiP-1 | MKLSLVAAMLLLLSLVAAMLLLLSAARA | 37 |
| Modified BiP-2 | MKLSLVAAMLLLLWVALLLLSAARA | 38 |
| Modified BiP-3 | MKLSLVAAMLLLLSLVALLLLSAARA | 39 |
| Modified BiP-4 | MKLSLVAAMLLLLALVALLLLSAARA | 40 |
| Gaussia | MGVKVLFALICIAVAEA | 41 |
| Native PPT1 Signal | MASPGCLWLLAVALLPWTCASRALQHL | 42 |
| Peptide (eSP) | | |
| Native PPT1 Signal | MASPGCLWLLAVALLPWTCASRALQHLAA | 43 |
| Peptide (eSP AA) | | |
| Native PPT1 Signal | MASPGSLWLLAVALLPWTCASRALQHL | 44 |
| Peptide C6S (eSP C6S) | | |
| Native PPT1 Signal | MASPGSLWLLAVALLPWTCASRALQHLAA | 45 |
| Peptide C6S (eSP C6S | | |
| AA) | | |
| Native TPP1 Signal | MGLQACLLGLFALILSGKC | 46 |
| Peptide | | |
| Native NAGLU Signal | MEAVAVAAAVGVLLLAGAGGAAGD | 47 |
| Peptide | | |

In some embodiments, the signal peptide sequence comprises amino acid sequence according to SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47.

One skilled in the art can readily derive a polynucleotide sequence encoding an amino acid sequence. Also, the polynucleotide sequence can be codon optimized for expression in target cells using commercially available products. The present invention includes the polynucleotide sequence encoding the tagged protein. The polynucleotide sequence encoding the tagged protein may be operably linked to expression control sequences, e.g., a promoter that directs expression of the gene, a secretion signal peptide gene.

### Expressing the Tagged Protein

In one of more embodiments, the method of producing the tagged protein comprises transfected host cells, allowed the host cells to transiently express the tagged protein, and purifying the tagged protein. In one or more embodiments, the tagged protein is expressed in host cells. In one or more embodiments, the host cells comprise any one of Expi293F cells, PC12 cells, HAP1 cells, Chinese hamster ovary (CHO) cells, HeLa cells, human embryonic kidney (HEK) cells, mouse primary myoblasts, NIH 3T3 cells, Escherichia coli cells, baculovirus expression system (e.g., Sf9 cells, Sf21 cells), yeast (e.g., Saccharomyces cerevisiae) or variants thereof. In one or more embodiments, cell-free synthesis is used to express the tagged protein.

In one of more embodiments, the host cells are transfected using suitable vectors, wherein the vector comprises a virus (such as an adenovirus, a parvovirus (e.g., adeno-associated virus (AAV)), a vaccinia virus, a herpesvirus, a baculovirus and a retrovirus or lentivirus), a bacteriophage, a cosmid, a plasmid, a bacterial artificial chromosome (BAC), a fungal vector, a naked DNA, a DNA lipid complex, a naked RNA, An RNA lipid complex or other recombination vehicles used in the art which have been described for expression in a variety of eukaryotic and prokaryotic hosts, and may be used for gene therapy as well as for simple protein expression.

After expression and secretion, recombinant protein can be recovered and purified from the surrounding cell culture media using standard techniques. Alternatively, recombinant protein can be isolated and purified directly from cells, rather than the medium.

### Determining the Binding Efficiency of the Tagged Protein for Sortilin

In one or more embodiments, binding the tagged protein to sortilin can be confirmed by a pull-down assay. In one or more embodiments, the pull-down assay is performed using a commercially available co-immunoprecipitation kit. In one of more embodiments, the pull-down assay is performed using a sortilin domain or a full-length sortilin protein. In one or more embodiments, the sortilin domain comprises amino acid residues 78-755 of the full length sortilin amino acid sequence. In one or more embodiments, the method of pull-down assay comprises, immobilizing sortilin, incubating the tagged protein with the immobilized sortilin, washing to remove unbound protein and quantifying the proportion of sortilin-bound tagged protein.

### Measuring the Activity of Sortilin-Bound Tagged Protein

In one of more embodiments, the functionality of the sortilin-bound tagged protein is measured. In one or more embodiments, the activity of the sortilin-bound tagged protein may be measured by a microplate binding assay, wherein the steps for the assay comprises immobilizing sortilin to the plate, incubating the tagged protein with the immobilized sortilin, measuring activity of the sortilin-bound tagged protein.

### Determination of Sortilin-Mediated Cellular Uptake of the Tagged Protein

Sortilin-mediated cellular uptake of the tagged protein can be determined by incubating cells with the tagged protein and identifying the cellular tagged protein. In one or more embodiments, the method comprises quantifying the transported tagged protein. In one or more embodiments, immunofluorescence can be used to identify the cellular tagged protein. In one or more embodiments, immunofluorescence can be used to quantify the cellular tagged protein.

### Determination of Activity of Sortilin-Mediated Intracellularly Transported Tagged Protein

The activity of the tagged protein inside cell can be determined by incubating the cells with the tagged protein and measuring the activity of the tagged protein inside cell. In one or more embodiments, intracellular protein activity can be assayed measuring fluorescence signal.

### Delivering the Tagged Protein to a Patient

Another aspect of the present invention relates to a pharmaceutical formulation comprising the tagged protein and a pharmaceutically acceptable carrier.

Another aspect of the present invention relates to a method of treating a disease or disorder comprising administering the pharmaceutical formulation to a patient in need thereof. In one or more embodiments, the disease or disease comprises a lysosomal storage disorder and the therapeutic protein comprises a lysosomal enzyme. In some embodiments, the lysosomal storage disorder is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachomatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type C1, Niemann-Pick disease type C2, Schindler disease type I, and Schindler disease type II. In some embodiments, the lysosomal storage disorder is selected from the group consisting of activator deficiency, GM2-gangliosidosis; GM2-gangliosidosis, AB variant; alpha-mannosidosis (type 2, moderate form; type 3, neonatal, severe); beta-mannosidosis; aspartylglucosaminuria; lysosomal acid lipase deficiency; cystinosis (late-onset juvenile or adolescent nephropathic type; infantile nephropathic); Chanarin-Dorfman syndrome; neutral lipid storage disease with myopathy; NLSDM; Danon disease; Fabry disease; Fabry disease type II, late-onset; Farber disease; Farber lipogranulomatosis; fucosidosis; galactosialidosis (combined neuraminidase & beta-galactosidase deficiency); Gaucher disease; type II Gaucher disease; type III Gaucher disease; type IIIC Gaucher disease; Gaucher disease, atypical, due to saposin C deficiency; GM1-gangliosidosis (late-infantile/juvenile GM1-gangliosidosis; adult/chronic GM1-gangliosidosis); Globoid cell leukodystrophy, Krabbe disease (Late infantile onset; Juvenile Onset; Adult Onset); Krabbe disease, atypical, due to saposin A deficiency; Metachromatic Leukodystrophy (juvenile; adult); partial cerebroside sulfate deficiency; pseudoarylsulfatase A deficiency; metachromatic leukodystrophy due to saposin B deficiency; Mucopolysaccharidoses disorders: MPS I, Hurler syndrome; MPS I, Hurler-Scheie syndrome; MPS I, Scheie syndrome; MPS II, Hunter syndrome; MPS II, Hunter syndrome; Sanfilippo syndrome Type A/MPS IIIA; Sanfilippo syndrome Type B/MPS IIIB; Sanfilippo syndrome Type C/MPS IIIC; Sanfilippo syndrome Type D/MPS IIID; Morquio syndrome, type A/MPS IVA; Morquio syndrome, type B/MPS IVB; MPS IX hyaluronidase deficiency; MPS VI Maroteaux-Lamy syndrome; MPS VII Sly syndrome; mucolipidosis I, sialidosis type II; I-cell disease, Leroy disease, mucolipidosis II; Pseudo-Hurler polydystrophy/mucolipidosis type III; mucolipidosis IIIC/ML III GAMMA; mucolipidosis type IV; multiple sulfatase deficiency; Niemann-Pick disease (type B; type C1/chronic neuronopathic form; type C2; type D/Nova Scotian type); Neuronal Ceroid Lipofuscinoses: CLN6 disease-Atypical Late Infantile, Late-Onset variant, Early Juvenile; Batten-Spielmeyer-Vogt/Juvenile NCL/CLN3 disease; Finnish Variant Late Infantile CLN5; Jansky-Bielschowsky disease/Late infantile CLN2/TPP1 Disease; Kufs/Adult-onset NCL/CLN4 disease (type B); Northern Epilepsy/variant late infantile CLN8; Santavuori-Haltia/Infantile CLN1/PPT disease; Pompe disease (glycogen storage disease type II); late-onset Pompe disease; Pycnodysostosis; Sandhoff disease/GM2 gangliosidosis; Sandhoff disease/GM2 gangliosidosis; Sandhoff disease/GM2 Gangliosidosis; Schindler disease (type III/intermediate, variable); Kanzaki disease; Salla disease; infantile free sialic acid storage disease (ISSD); spinal muscular atrophy with progressive myoclonic epilepsy (SMAPME); Tay-Sachs disease/GM2 gangliosidosis; juvenile-onset Tay-Sachs disease; late-onset Tay-Sachs disease; Christianson syndrome; Lowe oculocerebrorenal syndrome; Charcot-Marie-Tooth type 4J, CMT4J; Yunis-Varon syndrome; bilateral temporooccipital polymicrogyria (BTOP); X-linked hypercalciuric nephrolithiasis, Dent-1; and Dent disease 2. In one or more embodiments, the disease or disorder comprises Fabry disease and the therapeutic protein comprises alpha-galactosidase A. In one or more embodiments, the disease or disorder comprises Pompe disease and the therapeutic protein comprises alpha-glucosidase. In some embodiments, the therapeutic protein is associated with a lysosomal storage disorder and the therapeutic protein is selected from the group consisting of GM2-activator protein; α-mannosidase; MAN2B1; lysosomal ß-mannosidase; glycosylasparaginase; lysosomal acid lipase; cystinosin; CTNS; PNPLA2; lysosome-associated membrane protein-2; α-galactosidase A; GLA; acid ceramidase; α-L-fucosidase; protective protein/cathepsin A; acid ß-glucosidase; GBA; PSAP; ß-galactosidase-1; GLB1; galactosylceramide ß-galactosidase; GALC; PSAP; arylsulfatase A; ARSA; α-L-iduronidase; iduronate 2-sulfatase; heparan N-sulfatase; N-α-acetylglucosaminidase; heparan acetyl CoA: α-glucosaminide acetyltransferase; N-acetylglucosamine 6-sulfatase; galactosamine-6-sulfate sulfatase; ß-galactosidase; hyaluronidase; arylsulfatase B; ß-glucuronidase; neuraminidase; NEU1; gamma subunit of N-acetylglucosamine-1-phosphotransferase; mucolipin-1; sulfatase-modifying factor-1; acid sphingomyelinase; SMPD1; NPC1; and NPC2. In one or more embodiments, the disease or disorder comprises Batten disease and the therapeutic protein comprises PPT1 (CLN1), TPP1 (CLN2), CTSD (CLN10), PGRN (CLN11), or CTSF (CLN13).

In one or more embodiments, the pharmaceutical formulation is administered intrathecally, intravenously, intracisternally, intracerebroventrically, intraocularly, intravitreally, retinally, subretinally, intramuscularly, subcutaneously, intracerebrally, surgically or intraparenchymally.

### Gene Therapy

Another aspect of the present invention is application in gene therapy. In one or more embodiments, a gene therapy composition comprises a gene therapy delivery system and the polynucleotide encoding the tagged protein. In one or more embodiments, the gene therapy delivery system comprises one or more of a vector, a liposome, a lipid-nucleic acid nanoparticle, an exosome, and a gene editing system. In one or more embodiments, the gene therapy delivery system comprises one or more of Clustered Regularly Interspaced Short Palindromic Repeat (CRISPR) associated protein 9 (CRISPR-Cas-9), Transcription activator-like effector nuclease (TALEN), or ZNF (Zinc finger protein). In one or more embodiments, the gene therapy delivery system comprises a promoter. In one or more embodiments, the gene therapy delivery system comprises a polynucleotide encoding a secretion signal peptide.

In one or more embodiments, the gene therapy delivery system comprises a viral vector. In one or more embodiments, the viral vector comprises one or more of an adenoviral vector, an adeno-associated viral vector, a lentiviral vector, a retroviral vector, a poxviral vector, or a herpes simplex viral vector. The viral vectors also may include additional elements for increasing expression and/or stabilizing the vector such as promoters (e.g., hybrid CBA promoter (CBh) and human synapsin 1 promoter (hSyn1)), a polyadenylation signals (e.g., Bovine growth hormone polyadenylation signal (bGHpolyA)), stabilizing elements (e.g., Woodchuck Hepatitis Virus (WHP) Posttranscriptional Regulatory Element (WPRE)) and/or an SV40 intron. In one or more embodiments, the viral vector comprises a viral polynucleotide operably linked to the polynucleotide encoding the tagged protein. In one or more embodiments, the viral vector comprises at least one inverted terminal repeat (ITR).

In one or more embodiments, the gene therapy composition comprises a pharmaceutically acceptable carrier. In one or more embodiments, the pharmaceutically acceptable carrier comprises a diluent, adjuvant, excipient, or vehicle with which the compound is administered.

Various aspects of the present invention relate to a method of treating a disease or disorder comprising administering the gene therapy composition to a patient in need thereof. In one or more embodiments, the gene therapy composition is administered to a patient having a lysosomal storage disorder and the polynucleotide sequence comprises a nucleotide sequence encoding a lysosomal enzyme. In one or more embodiments, the gene therapy composition is administered to a patient having Fabry disease and the polynucleotide sequence comprises a nucleotide sequence encoding alpha-galactosidase A. In one or more embodiments, the gene therapy composition is administered to a patient having Pompe disease and the polynucleotide sequence comprises a nucleotide sequence encoding alpha-glucosidase. In one or more embodiments, the gene therapy composition is administered to a patient having Batten disease and the polynucleotide sequence comprises a nucleotide sequence encoding PPT1 (CLN1), TPP1 (CLN2), CTSD (CLN10), PGRN (CLN11), or CTSF (CLN13). In one of more embodiments, the gene therapy composition is administered intrathecally, intravenously, intracisternally, intracerebroventrically, intraocularly, intravitreally, retinally, subretinally, intramuscularly, subcutaneously, intracerebrally, surgically or intraparenchymally.

### EXAMPLES

### Example-1

### [A] Molecular Cloning and Expression of GAA Tagged Protein

Two variants of GAA tagged protein were cloned into Amicus proprietary plasmid such that NT (SEQ No. 1) was inserted either on the 5′ or 3′ end of either a therapeutic protein, GAA (RefSeq: NM_000152.5), or an affinity tag, mCherry (RefSeq: MN781140.1), with a flexible 9 amino acid linker peptide, between the affinity tag and the protein of interest. At the 5′ end of each transcript is a secretion signal. Tagged proteins were expressed transiently in Expi293F cell line. The tagged protein was then purified.

### [B] Determining the Binding Efficiency of the GAA Tagged Protein for Sortilin.

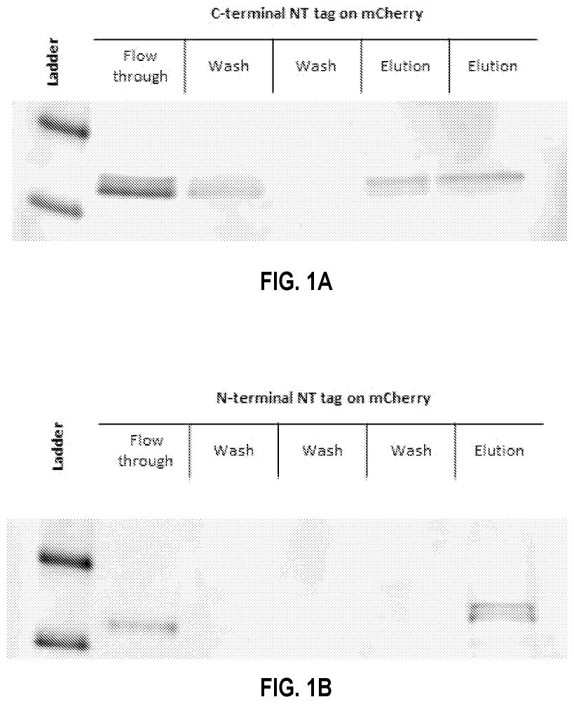

A pull-down assay for soluble sortilin (R&D systems, 3154-ST-050) was performed by covalently coupling the soluble sortilin domain to agarose beads via amine coupling chemistry using a commercially available co-immunoprecipitation kit (Thermo Fisher, 26149). Both, NT-GAA and GAA-NT were incubated independently with agitation at room temperature with the coupled beads for 2 hours. Following incubation, the bound proteins were eluted in fractions at low pH. The fractions and unbound protein elute were run on a standard SDS-Page reducing gel, and then proteins in the gel were transferred onto a nitrocellulose membrane for anti-mCherry staining (Invitrogen, M11217). After incubation with primary antibody, the membrane was washed and then stained with secondary antibody (Invitrogen, A21096), and then imaged. FIG. 1 shows that both GAA-NT (FIG. 1A) and NT-GAA (FIG. 1B) have high affinity for sortilin.

### [C] Measuring the Enzymic Activity of Sortilin-Bound GAA Tagged Protein.

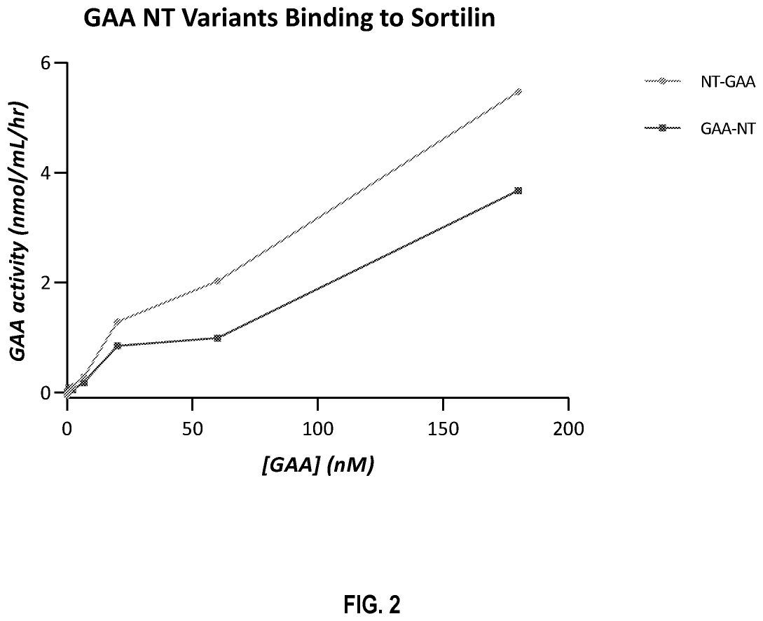

For a microplate binding assay, the extracellular soluble domain of sortilin, residues 78-755, was expressed in Expi293F cell line (using Expi293 Expression System; Thermo Fisher, A14635) with a C-terminal TwinStrep affinity tag. Harvests were collected after 5 days. Conditioned media was incubated with StrepTactin XT coated microplates (IBA, 2-4101-001) for 1 hour to immobilize sortilin to the plates. After several washes, wells coated with sortilin were incubated with varying doses of NT-GAA, GAA-NT or GAA diluted into PBS for 30 minutes. The unbound protein was then washed away. The α-D-glucopyranoside activity of the bound GAA tagged protein was measured using a fluorogenic substrate, 4-methylumbelliferyl-α-D-glucopyranoside, at pH 4.8. FIG. 2 shows α-D-glucopyranoside activity of the sortilin-bound NT-GAA and GAA-NT. The inflection point in both binding curves indicates bivalency of sortilin for neurotensin. GAA without the NT tag had no α-D-glucopyranoside activity indicating that the protein did not bind to sortilin.

### [D] Determining Sortilin Mediated Uptake of GAA Tagged Protein

COS7 cell line was used for immunofluorescence. Cells were seeded at approximately 40% confluency. The following day, cells were incubated with 200 nM of AT-GAA (Amicus enzyme replacement therapy), NT-GAA, or GAA-NT for 3 hours, either in the presence or absence of 10 mM mannose 6-phosphate in the uptake media. Following uptake, cells were washed with PBS, pH 7.4 and fixed with 4% formaldehyde in PBS for 10 minutes. Cells were then permeabilized with 0.1% Saponin in PBS for 5 minutes. Wells were incubated with anti-sortilin (Invitrogen, MA5-31437) and anti-GAA (Amicus produced) antibody for 1 hour. After washes with PBS, wells were incubated with fluorescent secondary antibodies for 1 hour (Thermo, A32723 and A11012), and then washed prior to imaging. DAPI was added for DNA staining in the final wash prior to imaging.

### [E] Intracellular Enzyme Activity of NT-Tagged GAA

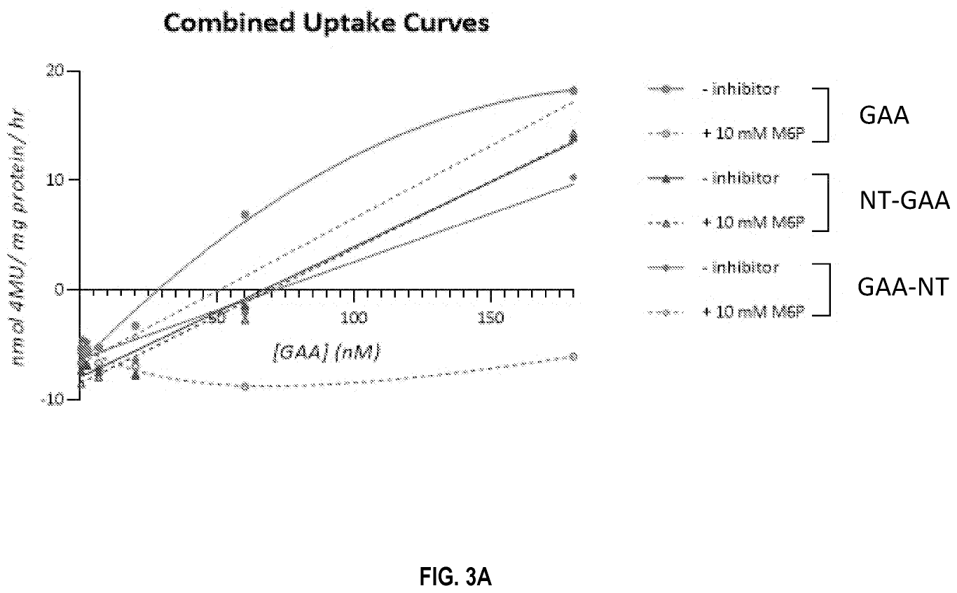

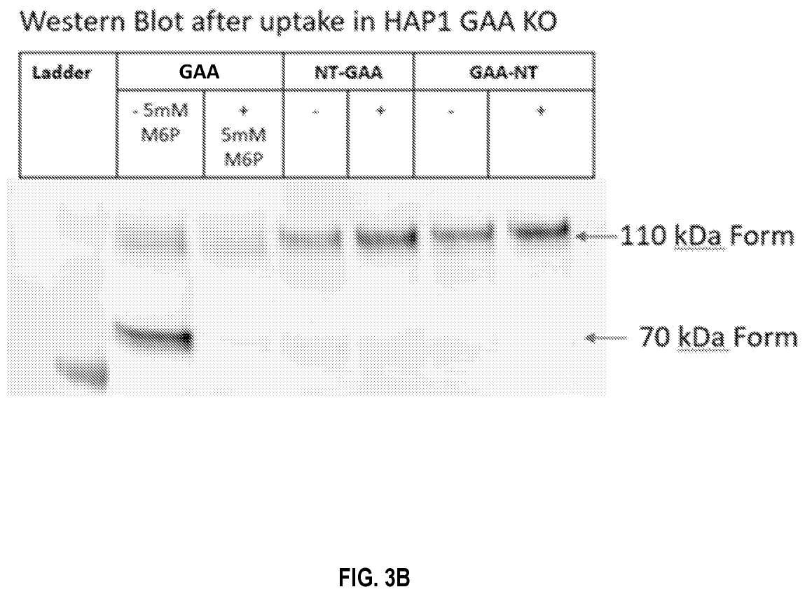

HAP1 GAA KO cell line was used for cell uptake assays. Cells were seeded in tissue cultured plates at approximately 40% confluency. The following day, the cells were incubated with varying concentrations of AT-GAA (Amicus enzyme replacement therapy), NT-GAA, or GAA-NT for 18 hours, either in the presence or absence of 10 mM mannose 6-phosphate in the uptake media. After 18 hours, the cells were washed several times with PBS, pH 7.4, and lysed with 0.25% Triton X-100 in dH₂O. α-D-glucopyranoside activity of intracellularly transported GAA tagged protein was measured by providing a fluorogenic substrate, 4-methylumbelliferyl-α-D-glucopyranoside, at pH 4.8 and measuring the 4-methylumbelliferone cleavage product. Cell lysates was run on standard SDS-page under reducing conditions followed by Western Blotting (Primary Ab: Abcam, ab137068; Secondary Ab: Invitrogen, SA535571). FIG. 3A shows that mannose-6-phosphate did not affect α-D-glucopyranoside activity in NT-GAA and GAA-NT incubated cells. Similarly, FIG. 3B shows that mannose-6-phosphate did not affect uptake of NT-GAA and GAA-NT. In conclusion, uptake of GAA tagged proteins is independent of CI-MPR and likely mediated by sortilin. Also, intracellularly transported GAA tagged proteins retain α-D-glucopyranoside activity.

What is claimed is:

1. A neurotensin peptide amino acid sequence comprising at least 70% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13, such that the amino acid sequence is not SEQ ID NO:1.

2. A neurotensin peptide amino acid sequence comprising at least 90% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13, such that the amino acid sequence is not SEQ ID NO:1.

3. A neurotensin peptide amino acid sequence comprising SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

4. A polynucleotide comprising a nucleotide sequence encoding the neurotensin peptide of any of claims 1-3.

5. A neurotensin polynucleotide comprising at least 40% nucleotide sequence identity to SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, such that the resulting polypeptide is not SEQ ID NO:1.

6. A neurotensin polynucleotide comprising at least 70% nucleotide sequence identity to SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, such that the resulting polypeptide is not SEQ ID NO:1.

7. A neurotensin polynucleotide comprising at least 97% nucleotide sequence identity to SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, such that the resulting polypeptide is not SEQ ID NO:1.

8. A neurotensin polynucleotide comprising SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

9. A sortilin propeptide amino acid sequence comprising at least 70% sequence identity to SEQ ID NO: 48, such that the amino acid sequence is not SEQ ID NO: 48.

10. A sortilin propeptide amino acid sequence comprising at least 90% sequence identity to SEQ ID NO: 48, such that the amino acid sequence is not SEQ ID NO:48.

11. A polynucleotide comprising a nucleotide sequence encoding the sortilin propeptide of claim 9 or 10.

12. A sortilin propeptide polynucleotide comprising at least 40% nucleotide sequence identity to SEQ ID NO: 49.

13. A sortilin propeptide polynucleotide comprising at least 70% nucleotide sequence identity to SEQ ID NO: 49.

14. A sortilin propeptide polynucleotide comprising at least 97% nucleotide sequence identity to SEQ ID NO: 49.

15. A sortilin propeptide polynucleotide comprising SEQ ID NO: 49.

16. A tagged protein comprising a tag and a therapeutic protein, wherein the tag comprises a neurotensin peptide and/or a sortilin propeptide.

17. The tagged protein of claim 16, wherein the neurotensin peptide comprises a polypeptide that is at least 70% identical to SEQ ID NO: 1.

18. The tagged protein of claim 16, wherein the neurotensin peptide comprises a polypeptide that is at least 90% identical to SEQ ID NO: 1.

19. The tagged protein of claim 16, wherein the neurotensin peptide comprises SEQ ID NO: 1.

20. The tagged protein of claim 16, wherein the neurotensin peptide comprises a polypeptide that is at least 70% identical to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

21. The tagged protein of claim 16, wherein the neurotensin peptide comprises a polypeptide that is at least 90% identical to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

22. The tagged protein of claim 16, wherein the neurotensin peptide comprises SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

23. The tagged protein of claim 16, wherein a polynucleotide encoding the neurotensin peptide comprises at least 40% sequence identity to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

24. The tagged protein of claim 16, wherein a polynucleotide encoding the neurotensin peptide comprises at least 70% sequence identity to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

25. The tagged protein of claim 16, wherein a polynucleotide encoding the neurotensin peptide comprises at least 97% sequence identity to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

26. The tagged protein of claim 16, wherein a polynucleotide encoding the neurotensin peptide comprises any one of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26.

27. The tagged protein of claim 16, wherein the sortilin propeptide comprises a polypeptide that is at least 70% identical to SEQ ID NO: 48.

28. The tagged protein of claim 16, wherein the sortilin propeptide comprises a polypeptide that is at least 90% identical to SEQ ID NO: 48.

29. The tagged protein of claim 16, wherein a polynucleotide encoding the sortilin propeptide comprises at least 40% sequence identity to SEQ ID NO: 50.

30. The tagged protein of claim 16, wherein a polynucleotide encoding the sortilin propeptide comprises at least 70% sequence identity to SEQ ID NO: 50.

31. The tagged protein of claim 16, wherein a polynucleotide encoding the sortilin propeptide comprises at least 97% sequence identity to SEQ ID NO: 50.

32. The tagged protein of claim 16, wherein a polynucleotide encoding the sortilin propeptide comprises SEQ ID NO: 50.

33. The tagged protein of any one of claims 16-32, wherein the therapeutic protein comprises a lysosomal protein.

34. The tagged protein of any one of claims 16-32, wherein the therapeutic protein comprises, alpha-galactosidase, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, and other Batten-related proteins, or an enzymatically active fragment thereof.

35. The Batten-related protein of claim 34, wherein the Batten-related protein comprises palmitoyl protein thioesterase 1 (PPT1) (CLN1), tripeptidyl peptidase 1 (TPP1) (CLN2), Cathepsin D (CTSD) (CLN10), progranulin (PGRN) (CLN11), and cathepsin F (CTSF) (CLN13).

36. The tagged protein of any of claims 16-32, wherein the therapeutic protein comprises alpha-glucosidase (GAA).

37. The tagged protein of any of claims 16-36, wherein the tagged protein comprises one or more of an affinity tag, a linker peptide, and a secretion signal peptide.

38. A polynucleotide comprising a nucleotide sequence encoding the tagged protein of any one of claims 16-37.

39. A method of producing the tagged protein of any one of claims 16-38, the method comprising expressing the tagged protein, and purifying the tagged protein.

40. The method of claim 39, wherein the tagged protein is expressed in Expi293F cells, PC12 cells, COS7 cell, HAP1 cells, Chinese hamster ovary (CHO) cells, HeLa cells, human embryonic kidney (HEK) cells, mouse primary myoblasts, NIH 3T3 cells, Escherichia coli cells, Sf9 cells, Sf21 cells, Saccharomyces cerevisiae or a variant thereof.

41. A method of determining cellular uptake for the tagged protein of any one of claims 16-38, the method comprising culturing cells, incubating the cells with the tagged protein, and identifying the intracellularly transported tagged protein.

42. The method of claim 41 comprises determining functionality of the intracellularly transported tagged protein.

43. The method of claim 41 or 42 comprises quantifying the intracellularly transported tagged protein.

44. The method of claims 41-43, wherein the cells are Expi293F cells, PC12 cells, COS7 cell, HAP1 cells, Chinese hamster ovary (CHO) cells, HeLa cells, human embryonic kidney (HEK) cells, mouse primary myoblasts, NIH 3T3 cells, Escherichia coli cells, Sf9 cells, Sf21 cells, yeast cells or a variant thereof.

45. A pharmaceutical formulation comprising:
the tagged protein of any one of claims 16-38; and
a pharmaceutically acceptable carrier.

46. A method of treating a disease or disorder comprising administering the pharmaceutical formulation of claim 45 to a patient in need thereof.

47. The method of claim 46, wherein the disease or disorder is Fabry disease and the therapeutic protein comprises alpha-galactosidase A.

48. The method of claim 46, wherein the disease or disorder is Pompe disease and the therapeutic protein comprises alpha-glucosidase A.

49. The method of claim 46, wherein the disease or disorder is Batten disease and the therapeutic protein comprises palmitoyl protein thioesterase 1 (PPT1) (CLN1), tripeptidyl peptidase 1 (TPP1) (CLN2), Cathepsin D (CTSD) (CLN10), progranulin (PGRN) (CLN11) and cathepsin F (CTSF) (CLN13).

50. The method of any one of claims 46-49, wherein the pharmaceutical formulation is administered intrathecally, intravenously, intracisternally, intracerebroventrically, intraocularly, intravitreally, retinally, subretinally, intramuscularly, subcutaneously, intracerebrally, surgically or intraparenchymally.

51. A gene therapy composition comprising:
a gene therapy delivery system; and
the polynucleotide encoding the tagged protein of any one of claims 16-38.

52. The gene therapy composition of claim 51, wherein the gene therapy delivery system comprises one or more of a vector, a liposome, a lipid-nucleic acid nanoparticle, an exosome, and a gene editing system.

53. The gene therapy composition of claim 51, wherein the gene therapy delivery system comprises one or more of Clustered Regularly Interspaced Short Palindromic Repeat (CRISPR) associated protein 9 (CRISPR-Cas-9), Transcription activator-like effector nuclease (TALEN), or ZNF (Zinc finger protein).

54. The gene therapy composition of claim 51 or 52, wherein the gene therapy delivery system comprises a viral vector.

55. The gene therapy composition of claim 54, wherein the viral vector comprises one or more of an adenoviral vector, an adeno-associated viral vector, a lentiviral vector, a retroviral vector, a poxviral vector, or a herpes simplex viral vector.

56. The gene therapy composition of claim 54 or 55, wherein the viral vector comprises a viral polynucleotide operably linked to the polynucleotide encoding the tagged protein.

57. The gene therapy composition of any one of claims 54-56, wherein the viral vector comprises at least one inverted terminal repeat (ITR).

58. The gene therapy composition of any one of claims 54-57, wherein the viral vector comprises one or more of an SV40 intron, a polyadenylation signal, or a stabilizing element.

59. The gene therapy composition of any one of claims 51-58, wherein the gene therapy delivery system comprises a promoter.

60. The gene therapy composition of any one of claims 51-59, wherein the gene therapy delivery system comprises a polynucleotide encoding a secretion signal peptide.

61. The gene therapy composition of any one of claims 51-60 comprises a pharmaceutically acceptable carrier.

62. A method of treating a disease or disorder comprising administering the gene therapy composition formulation of claim 61 to a patient in need thereof.

63. The method of claim 62, wherein the disease or disorder is Fabry disease and the polynucleotide sequence comprises a nucleotide sequence encoding alpha-galactosidase A.

64. The method of claim 62, wherein the disease or disorder is Pompe disease and the polynucleotide sequence comprises a nucleotide sequence encoding alpha-glucosidase.

65. The method of claim 62, wherein the disease or disorder is Batten disease and the polynucleotide sequence comprises a nucleotide sequence encoding palmitoyl protein thioesterase 1 (PPT1) (CLN1), tripeptidyl peptidase 1 (TPP1) (CLN2), Cathepsin D (CTSD) (CLN10), progranulin (PGRN) (CLN11) and cathepsin F (CTSF) (CLN13).

66. The method of any one of claims 61-65, wherein the gene therapy composition is administered intrathecally, intravenously, intracisternally, intracerebroventrically, intraocularly, intravitreally, retinally, subretinally, intramuscularly, subcutaneously, intracerebrally, surgically or intraparenchymally.


SEQUENCE LISTING (49 sequences)

SEQ ID NO: 1 - protein, 13 aa, Homo sapiens
ELYENKPRRPYIL

SEQ ID NO: 2 - protein, 13 aa, synthetic construct
ELYENKPRRPFIL

SEQ ID NO: 3 - protein, 13 aa, synthetic construct
ELYENKPRRPYIN

SEQ ID NO: 4 - protein, 13 aa, synthetic construct
ELYENKPRRPYID

SEQ ID NO: 5 - protein, 13 aa, synthetic construct
ELYENKPRRPYIE

SEQ ID NO: 6 - protein, 13 aa, synthetic construct
TLYENKPRRPYIL

SEQ ID NO: 7 - protein, 13 aa, synthetic construct
PLYENKPRRPYIL

SEQ ID NO: 8 - protein, 13 aa, synthetic construct
ELYEDKPRRPYIL

SEQ ID NO: 9 - protein, 13 aa, synthetic construct
ELRENKPRRPYIL

SEQ ID NO: 10 - protein, 13 aa, synthetic construct
ELYENKPRRGYIL

SEQ ID NO: 11 - protein, 13 aa, synthetic construct
ELYENKPRRPIIL

SEQ ID NO: 12 - protein, 13 aa, synthetic construct
ELYENKPARPYIL

SEQ ID NO: 13 - protein, 13 aa, synthetic construct
ELYENKPRAPYIL

SEQ ID NO: 14 - DNA, 39 bp, Homo sapiens
  source 1..39: mol_type = unassigned DNA
gagctgtacgagaacaagcccagacgcccctacatcctg

SEQ ID NO: 15 - DNA, 39 bp, synthetic construct
  source 1..39: mol_type = other DNA
gagctgtacgagaacaagcccagacgccccttcatcctg

SEQ ID NO: 16 - DNA, 39 bp, synthetic construct
  source 1..39: mol_type = other DNA
gagctgtacgagaacaagcccagacgcccctacatcaac

SEQ ID NO: 17 - DNA, 39 bp, synthetic construct
  source 1..39: mol_type = other DNA
gagctgtacgagaacaagcccagacgcccctacatcgac

SEQ ID NO: 18 - DNA, 39 bp, synthetic construct
  source 1..39: mol_type = other DNA
gagctgtacgagaacaagcccagacgcccctacatcgag

SEQ ID NO: 19 - DNA, 39 bp, synthetic construct
  source 1..39: mol_type = other DNA
accctgtacgagaacaagcccagacgcccctacatcctg

SEQ ID NO: 20 - DNA, 39 bp, synthetic construct
  source 1..39: mol_type = other DNA
cccctgtacgagaacaagcccagacgcccctacatcctg

SEQ ID NO: 21 - DNA, 39 bp, synthetic construct
  source 1..39: mol_type = other DNA
gagctgtacgacaacaagcccagacgcccctacatcctg

SEQ ID NO: 22 - DNA, 39 bp, synthetic construct
  source 1..39: mol_type = other DNA
gagctgagagagaacaagcccagacgcccctacatcctg

SEQ ID NO: 23 - DNA, 39 bp, synthetic construct
  source 1..39: mol_type = other DNA
gagctgtacgagaacaagcccagacgcggctacatcctg

SEQ ID NO: 24 - DNA, 39 bp, synthetic construct
  source 1..39: mol_type = other DNA
gagctgtacgagaacaagcccagacgccccatcatcctg

SEQ ID NO: 25 - DNA, 39 bp, synthetic construct
  source 1..39: mol_type = other DNA
gagctgtacgagaacaagcccgcccgcccctacatcctg

SEQ ID NO: 26 - DNA, 39 bp, synthetic construct
  source 1..39: mol_type = other DNA
gagctgtacgagaacaagcccagagccccctacatcctg

SEQ ID NO: 27 - protein, 9 aa, synthetic construct
RPRAVPTQA

SEQ ID NO: 28 - protein, 9 aa, synthetic construct
GGGGSGGGG

SEQ ID NO: 29 - protein, 5 aa, synthetic construct
GGGGS

SEQ ID NO: 30 - protein, 9 aa, synthetic construct
GGGSGGGGS

SEQ ID NO: 31 - protein, 9 aa, synthetic construct
GGGGSGGGS

SEQ ID NO: 32 - protein, 9 aa, synthetic construct
GGSGSGSTS

SEQ ID NO: 33 - protein, 10 aa, synthetic construct
GGGGSGGGGS

SEQ ID NO: 34 - protein, 12 aa, synthetic construct
GGGGSGSGGGGS

SEQ ID NO: 35 - protein, 9 aa, synthetic construct
RPRAVPTQA

SEQ ID NO: 36 - protein, 18 aa, Homo sapiens
MKLSLVAAMLLLLSAARA

SEQ ID NO: 37 - protein, 28 aa, synthetic construct
MKLSLVAAMLLLLSLVAAMLLLLSAARA

SEQ ID NO: 38 - protein, 25 aa, synthetic construct
MKLSLVAAMLLLLWVALLLLSAARA

SEQ ID NO: 39 - protein, 26 aa, synthetic construct
MKLSLVAAMLLLLSLVALLLLSAARA

SEQ ID NO: 40 - protein, 26 aa, synthetic construct
MKLSLVAAMLLLLALVALLLLSAARA

SEQ ID NO: 41 - protein, 17 aa, Gaussia princeps
MGVKVLFALICIAVAEA

SEQ ID NO: 42 - protein, 27 aa, Homo sapiens
MASPGCLWLLAVALLPWTCASRALQHL

SEQ ID NO: 43 - protein, 29 aa, synthetic construct
MASPGCLWLLAVALLPWTCASRALQHLAA

SEQ ID NO: 44 - protein, 27 aa, synthetic construct
MASPGSLWLLAVALLPWTCASRALQHL

SEQ ID NO: 45 - protein, 29 aa, synthetic construct
MASPGSLWLLAVALLPWTCASRALQHLAA

SEQ ID NO: 46 - protein, 19 aa, Homo sapiens
MGLQACLLGLFALILSGKC

SEQ ID NO: 47 - protein, 24 aa, Homo sapiens
MEAVAVAAAVGVLLLAGAGGAAGD

SEQ ID NO: 48 - protein, 44 aa, Homo sapiens
QDRLDAPPPPAAPLPRWSGPIGVSWGLRAAAAGGAFPRGGRWRG

SEQ ID NO: 49 - DNA, 132 bp, Homo sapiens
  source 1..132: mol_type = unassigned DNA
caagatcgattagatgctccccctccgcccgctgccccccttccaagatggagcggtccaattggggtttcctgggggctgcgggcagcagctgcaggaggagcctttcctaggggcggtcgttggcgcgga